(19) United States
(12) Patent Application Publication
(10) Pub. No.: US 2013/0005819 A1
(43) Pub. Date: Jan. 3, 2013

(54) CONTACT LENS DISINFECTING SOLUTIONS

(75) Inventors: Bandarage Mahesh K Bandara, Sydney NSW (AU); Mark D.P. Wilcox, Sydney NSW (AU); Hua Zhu, Sydney NSW (AU)

(73) Assignee: Brien Holden Vision Institute, Sydney NSW (AU)

(21) Appl. No.: 13/497,242

(22) PCT Filed: Sep. 10, 2010
(86) PCT No.: PCT/AU2010/001178

§ 371 Date: Aug. 27, 2012

(30) Foreign Application Priority Data
Sep. 21, 2009 (AU) 2009904553

(51) Int. Cl. A01N 37/52; C07C 309/08; A01P 1/00; C07C 257/18

(52) U.S. Cl. 514/625; 564/243; 562/112; 514/636; 514/635

(57) ABSTRACT

This invention relates to ophthalmic disinfecting and preserving compositions. More particularly, this invention relates to multi-purpose disinfecting solutions [MPDS] suitable for use in or with storage cases for treating multiple-use contact lenses and/or for preserving, disinfecting or packaging of contact and other ophthalmic lenses.

### FIELD OF THE INVENTION

This invention relates to ophthalmic disinfecting and preserving compositions. More particularly, this invention relates to multi-purpose disinfecting solutions [MPDS] suitable for use in or with storage cases for treating multiple-use contact lenses and/or for preserving, disinfecting or packaging of contact and other ophthalmic lenses.

### BACKGROUND

The increasing popularity of multiple-use soft contact lenses has led to a higher incidence rate of corneal infections (Dart et al. 1991; Cheng et al. 1999; Bourcier et al. 2003; Keay et al. 2007). Multiple-use contact lenses are typically worn during the day, removed before the user retires at night, placed in a lens case containing a disinfecting solution overnight and then removed from the case and re-fitted to the eyes for day-time wear. As multiple-use contact lenses are prone to contamination by bacteria, yeasts, fungi and other micro-organisms, and as they readily pick up oils and grime from the fingers or eyes of users, MPDS are used in lens cases to kill or inhibit the growth of a wide range of microbes and to remove grime and oils from the lens surfaces. The rising incidence of cornea infection among the users of multiple-use contact lenses has therefore raised serious concerns about the effectiveness of MPDS in current use.

MPDS performance for contact lenses is specified by various national and international standards, such as ISO Standard 14729 which stipulate minimal activity against nominated classes of micro-organisms under laboratory conditions. Thus according to ISO 14729, the disinfecting solution must meet the primary criteria of stand-alone procedures in which starting concentrations of specified bacteria and fungi (including yeasts) are reduced by mean values of not less than 3.0 and 1 logs respectively within a specified disinfection time (Standardization 2001). However, the literature shows that many available MPDS do not perform well under field conditions where multiple organisms are encountered. Indeed, it has been found that many commercially available products only have limited activity against clinical isolates of fungi and have minimal anti-amoebal activity under the laboratory tests (Cano-Parra et al. 1999; Dannelly and Waworuntu 2004; Hume et al. 2007; Ide et al. 2008; Dutot et al. 2009). Therefore, the need for a more effective MPDS for use with contact lenses is of great importance.

The anti-microbial compounds used or proposed for use in contact lens MPDS have been selected from known broad-spectrum anti-microbials that will be physiologically compatible with the eye. Multiple anti-microbial compounds have been used in an MPDS to enhance the spectrum of activity and MPDS are usually formulated with a physiologically compatible surfactant to assist in removing surface oils and grime from lens materials. Several cationic compounds have been widely used in antiseptic or disinfectant products as antimicrobial agents due to their intrinsic positive charge (Gilbert and Moore 2005; Epand and Epand 2009a; b; Grare et al. 2009) and have been tried in MPDS. Cationic antibacterial agents bind with high affinity to the negatively charged cell membranes of bacteria by displacing divalent cations in the membranes and causing the loss of essential cellular components (Gilbert and Moore 2005). Accordingly, cationic substances such as biguanides salts of alexidine, alexidine free base, salt of chlorhexidine, hexamethylene biguanides, and polymeric biguanides such as polyhexamethylene biguanide [PHMB]), quaternary ammonium compounds (i.e. polyquaternium-1 [POLYQUAD], chemical registry number 75345-27-6; and cetylpyridinium chloride [CPC]), and myristamidopropyl dimethylamine [ALDOX]) are used in MPDS as antimicrobial or disinfecting agents supplemented with other agents such as buffers, chelating agents, and surfactants (U.S. Pat. No. 6,063,745, U.S. Pat. No. 4,758,595, U.S. Pat. No. 4,407,791, U.S. Pat. No. 4,525,346, U.S. Pat. No. 4,836,986, U.S. Pat. No. 5,096,607, U.S. Pat. No. 7,578,996 B2, and WO 94/13774, European patent EP 1140224B1). In addition, other agents such as povidone-iodine, hydrogen peroxide, benzyl alcohol, indolicidins, ethoxylated alkyl glucoside, and non-amine polyethylenoxide are also used or suggested for MPDS as antimicrobial agents (U.S. Pat. No. 5,141,665; U.S. Pat. No. 6,482,799B1, U.S. Pat. No. 5,773,396, U.S. Pat. No. 5,405,878). Similarly other patents disclose the use of a composition containing dimethyldiallylammonium chloride homopolymer and strongly basic anionic exchange ammonium resins as antimicrobial agents for MPDS (U.S. Pat. No. 4,443,429 and U.S. Pat. No. 4,388,229 respectively).

More specifically, U.S. Pat. Nos. 6,063,745, 6,319,883, 6,482,781 and 6,586,377 disclose aqueous MPDS having 0.1-5 ppm of an antimicrobial biguanide such as PHMB, a surfactant comprising poly(oxyethylene)-poly(oxypropylene) block copolymer and a phosphate buffer sufficient to maintain the pH of the solution within a physiologically acceptable range. The use of a cellulosic component to adjust viscosity of the MPDS is also disclosed and U.S. Pat. No. 6,995,123 proposes the use of an ionic dissociating compound such as lauroylethylenediaminetriacetate as the surfactant, preferably in association with biguanide antimicrobials. U.S. Pat. No. 6,531,432 discloses a surfactant for use in MPDS selected from the group consisting of a polyethylene glycol fatty acid ester, an alkanolamide, an amide oxide, an ethoxylated alcohol and ethoxylated acid. U.S. Pat. No. 4,615,882 proposes the use of an organosilicon quaternary ammonium salt in an aqueous solution at concentrations between 0.001 and 0.5% (w/v). Various proposals have been published relating to the impregnation of anti-microbials in the plastic material from which lens cases are made so that these agents can leach into the MPDS (U.S. Pat. No. 2003/0176530 and U.S. Pat. No. 4,615,882).

In short, despite the wide search and experimentation with many known anti-microbials in MPDS, unsatisfactory performance of MPDS in the field is widely reported. The present invention is predicated on the discovery of a multi-purpose disinfecting solution that achieves satisfactory performance which is a contribution and provides an advantage over the prior art. Specifically, the inventors have found that disinfectant compounds belonging to the diamidine family are highly effective antimicrobials when used in a multi-purpose disinfecting solution.

### SUMMARY OF THE INVENTION

In one aspect, the present invention provides a multi-purpose disinfecting solution comprising a compound or any acceptable salt, functional variant, derivative or analog thereof wherein the compound has the formula (I):

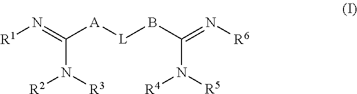

wherein A and B are each independently selected from the group consisting of C, N, O, optionally substituted C₃-C₁₂ cycloalkyl, optionally substituted C₆-C₁₈ aryl, optionally substituted C₁-C₁₈ heteroalkyl and optionally substituted C₁-C₁₈ heteroaryl; and

L, R¹, R², R³, R⁴, R⁵ and R⁶ are each independently selected from the group consisting of H, OH, NO₂, CN, NH₂, halogen, optionally substituted C₁-C₁₂ alkyl, optionally substituted C₂-C₁₂ alkenyl, optionally substituted C₂-C₁₂ alkynyl, optionally substituted C₃-C₁₂ cycloalkyl, optionally substituted C₃-C₁₂ cycloalkenyl, optionally substituted C₁-C₁₀ heteroalkyl, optionally substituted C₂-C₁₂ heterocycloalkyl, optionally substituted C₂-C₁₂ heterocycloalkenyl, optionally substituted C₆-C₁₈ aryl, optionally substituted C₁-C₁₈ heteroaryl, optionally substituted C₁-C₁₂ alkyloxy, optionally substituted C₁-C₁₂ alkyldioxy optionally substituted C₂-C₁₂ alkenyloxy, optionally substituted C₂-C₁₂ alkenyldioxy, optionally substituted C₂-C₁₂ alkynyloxy, optionally substituted C₂-C₁₂ alkynyldioxy, optionally substituted C₃-C₁₂ cycloalkyloxy, optionally substituted C₃-C₁₂ cycloalkenyloxy, optionally substituted C₁-C₁₀ heteroalkyloxy, optionally substituted C₁-C₁₂ heterocycloalkyloxy, optionally substituted C₁-C₁₂ heterocycloalkenyloxy, optionally substituted C₆-C₁₈ aryloxy, optionally substituted C₁-C₁₈ heteroaryloxy, optionally substituted C₁-C₁₂ alkylamino, SR⁷, SO₃H, SO₂NR⁷R⁸, SO₂R⁷, SONR⁷R⁸, SOR⁷, COR⁷, COOH, COOR⁷, CONR⁷R⁸, NR⁷COR⁸, NR⁷COOR⁸, NR⁷SO₂R⁸, NR⁷CONR⁸R⁹, NR⁷R⁸, OCOR⁷ and acyl; or

R¹ and R² together or R⁵ and R⁶ together may be fused to form a 5 or 6 membered cycloalkyl, heterocycloalkyl, aryl or heteroaryl ring each of which may be optionally substituted.

In another aspect, the present invention provides a lens case comprising a compound or any acceptable salt, functional variant, derivative or analog thereof wherein the compound has the formula (I) as described above.

In another aspect, the present invention provides a kit comprising a multi-purpose disinfecting solution as described above and instructions for using the multi-purpose disinfecting solution in a lens or storage case for multiple-use contact lenses.

In another aspect, the present invention provides a method of disinfecting or preserving a multiple-use contact lens comprising contacting the lens with a multi-purpose disinfecting solution as described above.

In yet another aspect, the present invention provides a method of disinfecting or preserving a multiple-use contact lens comprising contacting the lens with a compound or any acceptable salt, functional variant, derivative or analog thereof which has the formula (I) as described above.

In another aspect, the present invention provides a use of a multi-purpose disinfecting solution as described above for disinfecting or preserving a multiple-use contact lens.

In another aspect, the present invention provides a use of a compound or any acceptable salt, functional variant, derivative or analog thereof which has the formula (I) as described above for disinfecting or preserving a multiple-use contact lens.

In another aspect, the present invention provides use of a compound or any acceptable salt, functional variant, derivative or analog thereof in a multi-purpose disinfecting solution, wherein the compound has formula (I) as described above and wherein the compound is formulated to be at a maximum safe concentration in the multi-purpose disinfecting solution.

In another aspect, the present invention provides a compound or any acceptable salt, functional variant, derivative or analog thereof when used in a multi-purpose disinfecting solution or in a lens case, wherein the compound or any acceptable salt, functional variant, derivative or analog thereof has formula (I) as described above.

In an embodiment of the above aspects, the compound may have the formula (II):

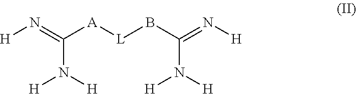

wherein A, B and L are as described above in respect of compounds having the formula (I).

In a preferred embodiment, A and B are each optionally substituted C₆ aryl.

In another preferred embodiment, L is optionally substituted C₁-C₁₂ alkyl, more preferably C₃-C₉ alkyl. Even more preferred are compounds of formula (I) or (II) wherein A and B are each optionally substituted C₆ aryl and L is C₃-C₉ alkyl.

The compound may be a diamidine. In a further embodiment, a derivative of the compound may be any isomer and/or tautomer including but not limited to organic acids, mineral acids and their salts. For example, an organic acid may be sulfonic acid or carboxylic acid.

In one embodiment, the compound may have the formula (III):

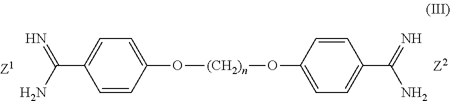

wherein n is 1, 2, 3, 4, 5, 6, 7, 8, 9, 10, 11 or 12, Z¹ and Z² are the same or different and are selected from an organic acid, its salt or a combination thereof. In yet another embodiment, Z¹ and Z² are selected from the group consisting of Isethionate, Methanesulfonate or both. In a preferred embodiment, Z¹ and Z² are the same.

In a preferred embodiment, n is 3, 4, 5, 6, 7, 8 or 9. Even more preferably, n is 6 (hexamidine; C₂₀H₂₆N₄O₂; Mr: 354.446; IUPAC name 4,4′-[hexane-1,6-diylbis(oxy)]dibenzenecarboximidamide)).

In yet a further embodiment, n is 6 and Z¹ and Z² are each isethionate.

In a further embodiment, the compound may be an anti-microbial agent.

In another embodiment, the compound or acceptable salt, functional variant, derivative or analog thereof may be present in the multi-purpose disinfecting solution or in the lens case. The compound or acceptable salt, functional variant, derivative or analog thereof may be present in and/or on the contact lens case. The compound or acceptable salt, functional variant, derivative or analog thereof may be embedded in the contact lens case. The structural component of the contact lens case may comprise any form of plastic or plastics. The compound or acceptable salt, functional variant, derivative or analog thereof may leach from the contact lens case or be present in an insert.

In yet another embodiment, the compound or acceptable salt, functional variant, derivative or analog thereof is active against Gram-positive and Gram-negative bacteria, yeasts and protozoa.

In another embodiment, the maximum safe concentration may be a concentration which is not toxic to ATCC L929 murine fibroblast according to ISO 10993-5 standard procedure for medical device cytotoxicity assessment.

In a further embodiment, the concentration is in a range between about 0.0001% (1 ppm) and about 0.1% (1000 ppm); between about 0.0005% (5 ppm) and about 0.005% (500 ppm); between about 0.001% (10 ppm) and about 0.025% (250 ppm); between about 0.005% (50 ppm) and about 0.02% (200 ppm); between about 0.01% (100 ppm) to about 0.02% (200 ppm); and about 0.01% (100 ppm).

In another embodiment, the multi-purpose disinfecting solution further comprises at least one surfactant and/or at least one buffer. The surfactant may be cationic, anionic or non-ionic.

In another embodiment, the solution or lens case may be formulated to comprise an effective amount of the compound to inhibit the growth of or to kill one or more microorganisms. The solution or lens case may further comprise an effective amount of a second compound to inhibit the growth of or to kill one or more microorganisms in combination with the first compound. The second compound may selected from the group comprising biguanides (i.e. salts of alexidine, alexidine free base, salt of chlorhexidine, hexamethylene biguanides and polymeric biguanides such as PHMB), myristamidopropyl dimethylamine, or polyquaternary ammonium compounds (i.e. POLYQUAD).

In another embodiment, the solution is isotonic or nearly isotonic wherein pH and tonicity of the solution is within a physiologically acceptable range.

In another embodiment, the solution may comprise any one or more enhancers. For example, the enhancer may be a surfactant and/or chelating agent. In one example, the chelating agent may be EDTA.

In another embodiment, the solution may further comprise any one or more of the following: buffering, osmotic, cleaning, wetting, and comfort enhancing agents. For example, one or more comfort enhancing and or wetting agent could be selected from the group comprising cellulose derivatives such as hydroxypropyl methyl cellulose, carboxymethyl cellulose, methyl cellulose, hydroxyethyl cellulose, dextran, gelatin, polyols, liquid such as glycerin, polyethylene glycol, polyethylene glycol, polysorbate, propylene glycol, polyvinyl alcohol, povidone (polyvinyl pyrrolidone) and copolymers such as EO/PO block copolymers.

The solution may comprise one or more of the surfactants and one or more of the comfort enhancing and/or wetting agents as described herein.

In another embodiment, the solution comprises:
(i) hexamidine diisethionate in a concentration of between about 50 ppm and about 200 ppm as the primary antiseptic at an effective and safe concentration,
(ii) a neutral or non-ionic surfactant such as poloxamine (registered under the trademark “Tetronic” BASF Corp) and Poloxamer (registered under the trademark “Pluronic” BASF Corp), and one or more of the following:
   (a) a pH buffer such as boric acid, sodium borate, sodium bicarbonate, citrate, TRIS and phosphate buffers and/or
   (b) a chelate such as ethylenediaminetetraacetic acid (EDTA) and its salts (ranging from about 0.01% to about 0.1% w/v), and/or
   (c) tonicity agents such as sodium chloride, and/or potassium chloride to adjust or maintain the osmolality of the solution within the range of about 220 to about 320 mOsm/kg.

### DEFINITIONS

In the specification and the claims the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

The term “maximum safe concentrations” means concentrations of any of the compounds described herein which are not toxic to ATCC L929 murine fibroblast according to ISO 10993-5 standard procedure for medical device cytotoxicity assessment.

As used herein, the term “multi-purpose disinfecting solution” or “MPDS” means a solution for use with contact lenses when not worn on the eye and whereby the solution has disinfecting and lens cleaning activities. One or more additional substances present in the MPDS may be active in disinfecting the lens, cleaning the lens or both. As a non-limiting example, the additional substances may be an enhancer such as a surfactant and/or chelating component, a buffering agent, an osmotic agent, a cleaning agent, a wetting agent or a comfort enhancing agent or any combination thereof.

The term “diamidine” means any compound that belongs to the diamidine family and consists of any molecule that comprises two amidines. One of ordinary skill in the art would understand that an “amidine” as used herein has the general formula RC(═NR)NR₂ where R can be carbon or hydrogen carbon. For instance, the simplest amidine is formamidine, HC(═NH)NH₂.

The term “functional variant” means any compound that would possess the base structure as defined by formula (I) and retain activity against microbes or microorganisms.

The term “physiological acceptable range” means any range of, for example, pH that would occur naturally in a human body.

The term “optionally substituted” as used throughout the specification denotes that the group may or may not be further substituted or fused (so as to form a polycyclic system), with one or more non-hydrogen substituent groups. In certain embodiments the substituent groups are one or more groups independently selected from the group consisting of halogen, ═O, ═S, —CN, —NO₂, —CF₃, —OCF₃, —OCHF₂, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —COOH, —COR¹¹, —C(O)OR¹¹, CONHR¹¹, NHCOR¹¹, NHCOOR¹¹, NHCONHR¹¹, C(═NOH)R¹¹, —SH, —SR¹¹, —OR¹¹, and acyl, wherein R¹¹ is H, C₂-C₁₂alkenyl, C₂-C₁₂ alkynyl, C₁-C₁₀ heteroalkyl, C₃-C₁₂ cycloalkyl, C₃-C₁₂ cycloalkenyl, C₁-C₁₂ heterocycloalkyl, C₁-C₁₀ heterocycloalkenyl, C₆-C₁₈ aryl, C₁-C₁₈ heteroaryl, and acyl.

“Alkyl” as a group or part of a group refers to a straight or branched aliphatic hydrocarbon group, such as a C₁-C₁₄ alkyl, a C₁-C₁₀ alkyl or a C₁-C₆ alkyl unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a —NH-Alkyl group, in which alkyl is as defined above. “Dialkylamino” means a —N(alkyl)₂ group, in which each alkyl may be the same or different and are each as defined herein for alkyl. The alkyl group may be a C₁-C₆ alkyl group. The group may be a terminal group or a bridging group.

“Arylamino” includes both mono-arylamino and di-arylamino unless specified. Mono-arylamino means a group of formula arylNH—, in which aryl is as defined herein. Di-arylamino means a group of formula (aryl)₂N— where each aryl may be the same or different and are each as defined herein for aryl. The group may be a terminal group or a bridging group.

“Acyl” means an alkyl-CO— group in which the alkyl group is as described herein. Examples of acyl include acetyl and benzoyl. The alkyl group may be a C₁-C₆ alkyl group. The group may be a terminal group or a bridging group.

“Alkenyl” as a group or part of a group denotes an aliphatic hydrocarbon group containing at least one carbon-carbon double bond and which may be straight or branched such as a group having 2-14 carbon atoms, 2-12 carbon atoms, or 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkoxy” refers to an —O-alkyl group in which alkyl is defined herein. The alkoxy may be a CᵣC₆alkoxy. Examples include, but are not limited to, methoxy and ethoxy. The group may be a terminal group or a bridging group.

“Alkenyloxy” refers to an —O-alkenyl group in which alkenyl is as defined herein. Preferred alkenyloxy groups are C₂-C₆ alkenyloxy groups. The group may be a terminal group or a bridging group.

“Alkynyloxy” refers to an —O-alkynyl group in which alkynyl is as defined herein. Preferred alkynyloxy groups are C₂-C₆ alkynyloxy groups. The group may be a terminal group or a bridging group.

“Alkoxycarbonyl” refers to an —C(O)—O-alkyl group in which alkyl is as defined herein. The alkyl group may be a C₁-C₆ alkyl group. Examples include, but not limited to, methoxycarbonyl and ethoxycarbonyl. The group may be a terminal group or a bridging group.

“Alkylsulfinyl” means a —S(O)-alkyl group in which alkyl is as defined above. The alkyl group is preferably a C₁-C₆ alkyl group. Exemplary alkylsulfinyl groups include, but not limited to, methylsulfinyl and ethylsulfinyl. The group may be a terminal group or a bridging group.

“Alkylsulfonyl” refers to a —S(O)₂-alkyl group in which alkyl is as defined above. The alkyl group may be a C₁-C₆ alkyl group. Examples include, but not limited to methylsulfonyl and ethylsulfonyl. The group may be a terminal group or a bridging group.

“Alkynyl” as a group or part of a group means an aliphatic hydrocarbon group containing a carbon-carbon triple bond and which may be straight or branched and may have from 2-14 carbon atoms, 2-12 carbon atoms, or 2-6 carbon atoms in the normal chain. Exemplary structures include, but are not limited to, ethynyl and propynyl. The group may be a terminal group or a bridging group.

“Alkylaminocarbonyl” refers to an alkylamino-carbonyl group in which alkylamino is as defined above. The group may be a terminal group or a bridging group.

“Cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

“Cycloalkenyl” means a non-aromatic monocyclic or multicyclic ring system containing at least one carbon-carbon double bond and may have from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group. The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

“Cycloalkylalkyl” means a cycloalkyl-alkyl-group in which the cycloalkyl and alkyl moieties are as previously described. Exemplary monocycloalkylalkyl groups include cyclopropylmethyl, cyclopentylmethyl, cyclohexylmethyl and cycloheptylmethyl. The group may be a terminal group or a bridging group.

“Halogen” represents fluorine, chlorine, bromine or iodine.

“Heterocycloalkyl” refers to a saturated or partially saturated monocyclic, bicyclic, or polycyclic ring containing at least one heteroatom selected from nitrogen, sulfur, oxygen. The heterocycloalkyl group may have from 1 to 3 heteroatoms in at least one ring. Each ring may be from 3 to 10 membered, such as 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. The group may be a terminal group or a bridging group.

“Heterocycloalkenyl” refers to a heterocycloalkyl as described above but containing at least one double bond. The group may be a terminal group or a bridging group.

“Heterocycloalkylalkyl” refers to a heterocycloalkyl-alkyl group in which the heterocycloalkyl and alkyl moieties are as previously described. Exemplary heterocycloalkylalkyl groups include (2-tetrahydrofuryl)methyl, (2-tetrahydrothiofuranyl)methyl. The group may be a terminal group or a bridging group.

“Heteroalkyl” refers to a straight- or branched-chain alkyl group that may have from 2 to 14 carbons, such as 2 to 10 carbons in the chain, one or more of which has been replaced by a heteroatom selected from S, O, P and N. Exemplary heteroalkyls include alkyl ethers, secondary and tertiary alkyl amines, amides, alkyl sulfides, and the like. The group may be a terminal group or a bridging group. As used herein reference to the normal chain when used in the context of a bridging group refers to the direct chain of atoms linking the two terminal positions of the bridging group.

“Aryl” as a group or part of a group denotes (i) an optionally substituted monocyclic, or fused polycyclic, aromatic carbocycle (ring structure having ring atoms that are all carbon) that may have from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Arylalkenyl” means an aryl-alkenyl-group in which the aryl and alkenyl are as previously described. Exemplary arylalkenyl groups include phenylallyl. The group may be a terminal group or a bridging group.

“Arylalkyl” means an aryl-alkyl-group in which the aryl and alkyl moieties are as previously described. Preferred arylalkyl groups contain a C₁₋₅ alkyl moiety. Exemplary arylalkyl groups include benzyl, phenethyl and naphthelenemethyl. The group may be a terminal group or a bridging group.

“Heteroaryl” either alone or as part of a group refers to groups containing an aromatic ring (such as a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

“Heteroarylalkyl” means a heteroaryl-alkyl group in which the heteroaryl and alkyl moieties are as previously described. The heteroarylalkyl groups may contain a lower alkyl moiety. Exemplary heteroarylalkyl groups include pyridylmethyl. The group may be a terminal group or a bridging group.

“Lower alkyl” as a group means, unless otherwise specified, an aliphatic hydrocarbon group which may be straight or branched having 1 to 6 carbon atoms in the chain, for example 1 to 4 carbons such as methyl, ethyl, propyl (n-propyl or isopropyl) or butyl (n-butyl, isobutyl or tertiary-butyl). The group may be a terminal group or a bridging group.

The term “acceptable salts” refers to salts that retain the desired biological activity of the above-identified compounds, and includey acceptable acid addition salts and base addition salts. Suitable acceptable acid addition salts of compounds of Formulae (I), (II) and (III) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable acceptable base addition salts of compounds of Formulae (I), (II) and (III) include metallic salts made from lithium, sodium, potassium, magnesium, calcium, aluminium, and zinc, and organic salts made from organic bases such as choline, diethanolamine, morpholine. Other examples of organic salts are: ammonium salts, quaternary salts such as tetramethylammonium salt; amino acid addition salts such as salts with glycine and arginine. Additional information on acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

### DETAILED DESCRIPTION OF THE INVENTION

The inventors have found that disinfectant compounds belonging to the diamidine family such as the compounds of Formulae (I), (II) and (III) are highly effective antimicrobials when used in MPDS. The inventors have found that the compounds of Formulae (I), (II) and (III) are highly effective antimicrobials when used in MPDS in contact lens cases together with commonly used surfactants and buffers. Moreover, the safety and efficacy of these disinfectants in relation to the human body have been widely tested and diamidine compounds are used as preservatives and biocides in cosmetics, personal-care products and topical pharmaceutical preparations. It appears that the potential of diamidines as anti-microbials in MPDS has been overlooked in the past because of misplaced complacency about the effectiveness of the very wide range of alternative, better known and more conventional antiseptic compounds.

Most generally, the present invention comprises use of a multi-purpose disinfecting solution comprising a compound or any acceptable salt, functional variant, derivative or analog thereof wherein the compound has the formula (I):

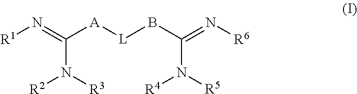

wherein A and B are each independently selected from the group consisting of C, N, O, optionally substituted C₃-C₁₂ cycloalkyl, optionally substituted C₆-C₁₈ aryl, optionally substituted C₁-C₁₈ heteroalkyl and optionally substituted C₁-C₁₈ heteroaryl; and

L, R¹, R², R³, R⁴, R⁵ and R⁶ are each independently selected from the group consisting of H, OH, NO₂, CN, NH₂, halogen, optionally substituted C₁-C₁₂ alkyl, optionally substituted C₂-C₁₂ alkenyl, optionally substituted C₂-C₁₂ alkynyl, optionally substituted C₃-C₁₂ cycloalkyl, optionally substituted C₃-C₁₂ cycloalkenyl, optionally substituted C₁-C₁₀ heteroalkyl, optionally substituted C₂-C₁₂ heterocycloalkyl, optionally substituted C₂-C₁₂ heterocycloalkenyl, optionally substituted C₆-C₁₈ aryl, optionally substituted C₁-C₁₈ heteroaryl, optionally substituted C₁-C₁₂ alkyloxy, optionally substituted C₁-C₁₂ alkyldioxy, optionally substituted C₂-C₁₂ alkenyloxy, optionally substituted C₂-C₁₂ alkenyldioxy, optionally substituted C₂-C₁₂ alkynyloxy, optionally substituted C₂-C₁₂ alkynyldioxy, optionally substituted C₃-C₁₂ cycloalkyloxy, optionally substituted C₃-C₁₂ cycloalkenyloxy, optionally substituted C₁-C₁₀ heteroalkyloxy, optionally substituted C₁-C₁₂ heterocycloalkyloxy, optionally substituted C₁-C₁₂ heterocycloalkenyloxy, optionally substituted C₆-C₁₈ aryloxy, optionally substituted C₁-C₁₈ heteroaryloxy, optionally substituted C₁-C₁₂ alkylamino, SR⁷, SO₃H, SO₃NR⁷R⁸, SO₂R⁷, SONR⁷R⁸, SOR⁷, COR⁷, COOH, COOR⁷, CONR⁷R⁸, NR⁷COR⁸, NR⁷COOR⁸, NR⁷SO₂R⁸, NR⁷CONR⁸R⁹, NR⁷R⁸, OCOR⁷, and acyl.

As contemplated herein, a lens case comprises a compound or any acceptable salt, functional variant, derivative or analog thereof wherein the compound has the formula (I) as described above.

There is also contemplation of use of a compound or any acceptable salt, functional variant, derivative or analog thereof in a multi-purpose disinfecting solution, wherein the compound has formula (I) as described above and wherein the compound is formulated to be at a maximum safe concentration in the multi-purpose disinfecting solution.

There is also contemplation of a kit comprising a multi-purpose disinfecting solution as described above and instructions for using the multi-purpose disinfecting solution in a lens or storage case for multiple-use contact lenses.

Also contemplated is a method of disinfecting or preserving a multiple-use contact lens comprising contacting the lens with either a multi-purpose disinfecting solution as described above or with a compound or any acceptable salt, functional variant, derivative or analog thereof which has the formula (I) as described above.

There is also contemplated a use of a multi-purpose disinfecting solution as described above or a compound or any acceptable salt, functional variant, derivative or analog thereof which has the formula (I) as described above for disinfecting or preserving a multiple-use contact lens.

In preferred embodiments of the methods and uses described above, the compound of formula (I) is hexamidine diisethionate.

A compound or any salt, variant, derivative or analog thereof when used in a multi-purpose disinfecting solution or in a lens case, formula (I) as described above is also contemplated.

Further contemplation of the compound may have the formula (II):

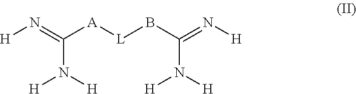

wherein A, B and L are as described above in respect of compounds having the formula (I).

In a preferred embodiment, A and B are each optionally substituted C₆ aryl.

In another preferred embodiment, L is optionally substituted C₁-C₁₂ alkyl, more preferably C₃-C₉ alkyl. Even more preferred are compounds of formula (I) or (II) wherein A and B are each optionally substituted C₆ aryl and L is C₃-C₉ alkyl.

Generally, the present invention comprises use of a diamidine compound or compounds, their salts, and derivatives as anti-microbials in or with contact lens cases, whether in MPDS or in the plastics from which lens cases—or inserts therefore—are manufactured. Diamidines are known aromatic compounds and have a broad spectrum of antimicrobial activity against a range of Gram-positive and Gram-negative bacteria, yeasts and protozoa (Wien et al. 1948; Perrine et al., 1995; Seal 2003; Grare et al. 2009). They are bipolar molecules consisting of two benzene rings connected by an alkyl chain and a protonated hydrophilic amidine group.

In general, the diamidine compounds useful in the present invention include compounds that correspond to those of formula (III):

(III)

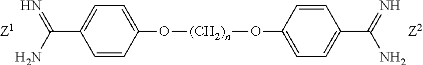

| Compound (value of n) | Z¹ and Z² |
|---|---|
| Propamidine (3) | Isethionate |
| Butamidine (4) | Methanesulfonate |
| Pentamidine (5) | Isethionate |
| Hexamidine (6) | Isethionate |
| Heptamidine (7) | Methanesulfonate |
| Octamidine (8) | Methanesulfonate |
| Nonamidine (9) | Methanesulfonate |

As used herein, diamidine derivatives include any isomers and tautomers of diamidine compounds including but not limited to organic acids, mineral acids and their salts, for example sulfonic acid, carboxylic acid, etc.

As contemplated herein, any compound that falls within the scope of a diamidine may be synthesised and used in working of the invention. As would be understood by the skilled person, throughout the synthesis of the compounds of Formula (I), for example, it may be necessary to employ a protecting group on the amino group and/or on the carboxyl group in order to reversibly preserve a reactive amino or carboxyl functionality while reacting other functional groups on the compound. In such a case, the free amino group and/or the free carboxyl groups of the compounds of Formula (I) can be liberated either by deprotection of the amino group followed by deprotection of the acid moieties or vice versa.

Examples of suitable amino protecting groups that may be used include formyl, trityl, phthalimido, trichloroacetyl, chloroacetyl, bromoacetyl, iodoacetyl, and urethane-type blocking groups such as benzyloxycarbonyl (‘CBz’), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4cyanobenzyloxycarbonyl, t-butoxycarbonyl (‘tBoc’), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxycarbonyl, cyclopentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (“FMOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual amino protecting group employed is not critical so long as the derivatised amino group is stable to the condition of subsequent reaction(s) and can be selectively removed as required without substantially disrupting the remainder of the molecule including any other amino protecting group(s). Preferred amino-protecting groups are t-butoxycarbonyl (Boc), and benzyloxycarbonyl (Cbz). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Theime Medical Pub., 2000.

Examples of carboxyl protecting groups that may be used include methyl, ethyl, n-propyl, i-propyl, p-nitrobenzyl, p-methylbenzyl, p-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′4,4′-tetramethoxybenzhydryl, t-butyl, t-amyl, trityl, 4-methoxytrityl, 4,4′-dimethoxytrityl, 4,4,′4″-trimethoxytrityl, 2-phenylprop-2-yl, trimethylsilyl, t-butyldimethylsilyl, phenacyl, 2,2,2-trichloroethyl, β-(di(n-butyl)methylsilyl)ethyl, p-toluenesulfonoethyl, 4-nitrobenzylsulfonoethyl, allyl, cinnamyl, 1-(trimethylsilylmethyl)prop-1-en-3-yl, and the like. Preferred carboxyl protecting groups are methyl and t-butyl. Further examples of these groups are found in: Greene, T. W. and Wuts, P. G. M., Protective Groups in Organic Synthesis, Second edition; Wiley-Interscience: 1991; McOmie, J. F. W. (ed.), Protective Groups in Organic Chemistry, Plenum Press, 1973; and Kocienski, P. J., Protecting Groups, Second Edition, Theime Medical Pub., 2000.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, formulae (I), (II) and (III) are intended to cover, where applicable, solvated as well as unsolvated forms of the compounds. Thus, each formula includes compounds having the indicated structure, including the hydrated as well as the non-hydrated forms.

In addition to compounds of the formulae (I), (II) and (III), the compounds of the various embodiments include acceptable salts, prodrugs, N-oxides and active metabolites of such compounds, and acceptable salts of such metabolites.

From another aspect of the present invention, the selected compounds of the formulae (I), (II) and (III) are preferably employed in concentrations which are not toxic to ATCC L929 murine fibroblast according to ISO 10993-5 standard procedure for medical device cytotoxicity assessment and such concentrations are referred to herein as the ‘maximum safe’ concentrations.

In order to have the desired antimicrobial effectiveness, diamidine compounds can be used at concentrations ranging between about 0.0001% (1 ppm) and about 0.1% (1000 ppm). Preferably, the diamidine compound comprises hexamidine diisethionate (herein referred to as ‘HD’ for convenience). It is desirable for a better biocompatibility that the HD concentration in MPDS is no more than 0.02% (200 ppm), more preferably no more than about 0.015% (150 ppm). While the minimum concentration of HD is that which makes it effective as a disinfectant, the practical minimum concentration is about 10 ppm, preferably about 50 ppm and most preferably about 100 ppm. Such minimums being subject to the above consideration of maximum safe concentration of the selected diamidine compound.

The basic active ingredient in formulations of the present invention comprises an effective concentration of a compound of the formulae (I), (II) and (III), either alone or in combination with other antimicrobial components at reduced concentrations, in an isotonic or nearly isotonic buffer system which maintains the pH and tonicity of the solution within a physiologically acceptable range. Furthermore, it has been discovered that a range of known cleaning enhancers such as surfactants and chelating components used in conventional MPDS are compatible with diamidines, for example EDTA can be added to the basic solution at conventional concentrations without adverse affect on diamidine activity. Similarly, known buffering, osmotic, cleaning, wetting, and comfort enhancing agents can be added to enhance the final formulation. The multi-purpose disinfecting solution may comprise at least one surfactant and/or at least one buffer. The surfactant may be cationic, anionic or non-ionic.

The solution may comprise any one or more enhancers. For example, the enhancer may be a surfactant and/or chelating component. In one example, the chelating agent may be EDTA. Non-limiting examples of comfort enhancing and or wetting agents include cellulose derivatives such as hydroxypropyl methyl cellulose, carboxymethyl cellulose, methyl cellulose, hydroxyethyl cellulose, dextran, gelatin, polyols, liquid such as glycerin, polyethylene glycol, polyethylene glycol, polysorbate, propylene glycol, polyvinyl alcohol, povidone (polyvinyl pyrrolidone) and copolymers such as EO/PO block copolymers.

As contemplated herein, the solution may comprise one or more of the surfactants and one or more of the comfort enhancing and/or wetting agents as described herein.

From another aspect, the present invention comprises a biocompatible aqueous disinfecting solution for use in lens cases, and lens cases containing such a solution, the aqueous solution being characterised in that it comprises hexamidine diisethionate in a concentration of between 50 and 200 ppm as the primary antiseptic at an effective and safe concentration, a neutral or non-ionic surfactant such as poloxamine (registered under the trademark “Tetronic” BASF Corp) and Poloxamer (registered under the trademark “Pluronic” BASF Corp), and one or more of the following: a pH buffer such as boric acid, sodium borate, sodium bicarbonate, citrate, TRIS and phosphate buffers and/or a chelate such as ethylenediaminetetraacetic acid (EDTA) and its salts (ranging from 0.01% to 0.1% w/v), and/or tonicity agents such as sodium chloride, and/or potassium chloride to adjust or maintain the osmolality of the solution within the range of about 220 to about 320 mOsm/kg.

From another aspect of the present invention, a secondary anti-microbial agent is employed together with the selected compound to broaden the activity spectrum or to present additional modes of antimicrobial action. Preferably, the secondary antimicrobial or microbials is/are used at a lower concentration relative to that of the diamidine (which is preferably HD) and may be an agent or agents conventionally used in MPDS. For example, the secondary anti-microbial agent may be selected from one or more of the following: biguanides (i.e. salts of alexidine, alexidine free base, salt of chlorhexidine, hexamethylene biguanides and polymeric biguanides such as PHMB), myristamidopropyl dimethylamine, or polyquaternary ammonium compounds (i.e. POLYQUAD).

As already noted, it is surprising that hexamidine diisethionate and related hexamidine derivatives (which are known anti-microbials) have not been previously used in MPDS but, from our trials, are significantly more effective as broad-spectrum antimicrobial agents in MPDS than the disinfecting agents in current use or disclosed in the prior art summarised above. Indeed, the clinical effectiveness and safety of HD has been demonstrated in several clinical trials unrelated to MPDS and with general wide usage in people of all ages (Budavari 1989). The nature of its (HD) prior use and the family of compounds here included as ‘hexamidine diisethionate and related amidine derivatives’ is considered below.

Hexamidine (C₂₀H₂₆N₄O₂; Mr: 354.446; IUPAC name 4,4′-[hexane-1,6-diylbis(oxy)]dibenzenecarboximidamide) is an aromatic diamidine antiseptic. The structure of hexamidine diisethionate, the most preferred anti-microbial diamidines in the current invention is shown below:

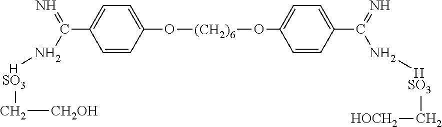

Molecular weight: 606.711, and Molecular formula: C₂₄H₃₈N₄O₁₀S₂

Hexamidine diisethionate (HD), derivative of diamidines currently used as a preservative and biocide at concentrations of 0.03% to 0.1% in cosmetics and personal-care products such as hair, nail, and skin-care products, as well as baby products [http://www.cosmeticdatabase.com] (Review 2007; Toxicol 2007). HD and its derivatives (0.0001% to 25% by weight of the composition) are useful for regulating the condition of mammalian keratinous tissue, particularly human skin tissue (Bissett 2004, US patent NO; 2004/0176273). Personal care compositions containing HD to treat conditions of mammalian keratinous tissue such as skin, hair, or nail are reported (U.S. Pat. No. 2008/0095732 A1). Similarly another patent describes a composition containing hexamidine and sugar amine and their combinations (0.1% to 10% by weight of the composition) for regulating mammalian keratinous tissue (US patent NO; 2007/7285570 B2). Patent 2004/0175343A1 discloses a novel composition of a skin care product with a hydrophobic barrier protectant which contains hexamidine and its salt derivatives (Osborne et al., US patent NO; 2004/0175343A1).

HD has been demonstrated to have antimicrobial properties against a range of Gram-positive and Gram-negative bacteria and Acanthamoeba strains (Wien et al. 1948; Brasseur et al. 1994; Vasseneix et al. 2006; Grare et al. 2009). An antiseptic eye drop (Desomedine, Bausch and Lomb) containing hexamidine isethionate (0.1%) has shown to be very active against both trophozoites and cysts of several strains of Acanthamoeba (Brasseur et al. 1994). Previously it has also been shown that HD in combination with neomycin was active during treatment of human keratitis with microbes resistant to propamidine (Brasseur et al. 1994). Perrine et al also suggested that replacing propamidine isethinoate with hexamidine in treatment of Acanthamoeba keratitis (Perrine et al. 1995). Moreover, combinations of PHMB and HD exert a synergistic effect against A. polyphaga keratitis in a rat model (Vasseneix et al. 2006). This combination shows higher antimicrobial activity than that of the individual substances. However, high concentrations of PHMB (0.02% or 200 ppm) and HD (0.1%) were used in the reported studies (Vasseneix et al. 2006). Another U.S. Pat. No. 4,505,924 disclosed that an effective pharmaceutical preparation for the topical treatment of acne in humans contains a combination of imazalil or acid addition salt and HD (range of from 100:1 to 10:1 parts by body weight) and found that the combination of HD and imazalil or an acid addition salt had an improved microbiocidal effect against acne-causing bacteria such as Propionibacterium acnes and Staphylococcus epidermis. Moreover, US patent 2007/0078118 A1 has shown that the combination of HD and N-(n-butyl)-1,2-benzisothiazolin or N-methyl-1,2-benzisothiazolin-3-one has a synergistic microbicidal effect against bacteria and yeast. In addition, there is a patent for disposable pre-moistened wipes containing HD (0.0005% to 10% by weight of said liquid) as an antimicrobial protease inhibitor (U.S. Pat. No. 6,207,596). Furthermore U.S. Pat. No. 6,287,584 discloses that hydrophilic sponges or non woven wipers containing HD have advantageous properties including a long-lasting antimicrobial effect (U.S. Pat. No. 6,287,584).

Propamidine (C₁₇H₂₀N₄O₂; Mr. 312.37), a derivative of diamidines, is an antiseptic and disinfectant and is also used to treat infections of the eye, such as conjunctivitis and Acanthamoeba infection (Wright et al. 1985). In addition, combinations of polymyxin B and dibromopropamidine isethionate exhibited synergistic inhibitory and bactericidal activity against a range of Gram-positive and Gram-negative bacteria (Richards and Xing 1994).

Pentamidine another derivative of diamidines also has an antimicrobial activity against Acanthamoeba infection (John et al. 1990; Alizadeh et al. 1997). Additionally, pentamidine has good clinical activity in treating Leishmaniasis, sleeping sickness caused by different strains of Trypanosoma, and yeast infections caused by the organism Candida albicans (http://en.wikipedia.org/wiki/Pentamidine).

Having portrayed the nature of the present invention, particular examples will now be described. However, those skilled in the art will appreciate that many variations and modifications can be made to the examples without departing from the scope and/or spirit of the invention as outlined above. The examples are directed to various embodiments of the invention and thus should not be interpreted to limit the scope or define the broadest scope of the invention.

### EXAMPLES

### Example 1

The solution compositions were prepared according to the following formulation in distilled water and sterilized by filtering through a 0.22 micron filter.

TABLE 1.1

| Ingredients | w/v |
|---|---|
| Hexamidine diisethionate (HD) | 0.01% |
| Polyhexamethylene biguanide (PHMB) | 1.5 ppm |
| Poloxamine (Pluronic F87) | 0.05% |
| Na₂ EDTA | 0.05% |
| Potassium dihydrogen orthophosphate (KH₂PO₄) | 0.2% |
| di-sodium hydrogen orthophosphate (anhydrous | 1.15% |
| Na₂HPO₄) | |
| Potassium chloride (KCl) | 0.2% |
| Sodium chloride (NaCl) | 0.8% |
| pH | 7.6 |

In this example, the effectiveness of HD in combination with 1.5 ppm of PHMB was evaluated using a phosphate buffer system. An aqueous contact lens disinfection solution in Example I (Table 1.1) was prepared by mixing together the above ingredients.

The bactericidal activity of the formulation was examined in accordance with the Stand-alone test procedure recommended in ISO standard for contact lens disinfection solutions (ISO/14729) by using ISO panel microorganisms Staphylococcus aureus ATCC 6538, Pseudomonas aeruginosa ATCC 9027 and Serratia marcescens ATCC 13880. In addition, Acanthamoeba polyphaga ROS (MCC 3315) (at the concentration of 10⁵ cells per mL) was also used as a challenge strain. Each challenge strain was exposed to said solution at room temperature for 6 hours.

The ability of the formulation to resist neutralization by organic soil (a mixture of serum and killed yeast cells) was determined by exposing each challenge bacterium to the solution in the presence of fetal bovine serum (1%) and killed yeast cells (Saccharomyces cerevisiae at a concentration of 10⁶ CFU/mL).

The antimicrobial efficacy of the test solution of Example 1 is illustrated in Table 1. 2.

TABLE 1.2

| | Log reduction at 6 hours | |
|---|---|---|
| | No | With |
| Microorganism | organic soil | organic soil |
| Staphylococcus aureus ATCC 6538 | 5.99 | 6.10 |
| Pseudomonas aeruginosa ATCC 9027 | 5.87 | 5.94 |
| Serratia marcescens ATCC 13880 | 4.57 | 3.25 |
| Acanthamoeba polyphaga ROS (MCC 3315) | 3.1 | ND |
| (trophozoites) | | |
| Acanthamoeba polyphaga ROS (MCC 3315) | 2.8 | ND |
| (cysts) | | |

ND = not determined

As can be seen, the formulation containing HD completely killed challenge strains S. aureus and P. aeruginosa even in the presence of organic soil. The formulation with no organic soil reduced the level of the difficult-to-kill organism S. marcescens by 4.57 log units. In the presence of organic soil, the solution reduced the amount of S. marcescens by 3.25 log units, exceeding the ISO criterion of at least 3-log reduction of each challenge bacterial strain. To be considered effective in the ISO Stand-alone test, there must be at least 3-log reduction in number of bacteria and f-log reduction in number of fungi for each challenge strain. Furthermore, the solution reduced the number of another difficult-to-kill organism A. polyphaga by 3.1-log units for trophozoites and 2.8-log units for cysts. Currently, there is no requirement for contact lens disinfection solution with amoebicidal activity in ISO standard.

### Example 2

TABLE 2.1

w/v

| Ingredients | 2A | 2B |
|---|---|---|
| Hexamidine diisethionate (HD) | 0.01% | 0.01% |
| Polyhexamethylene biguanide (PHMB) | 1.5 ppm | 1.0 ppm |
| Poloxamine (Pluronic F87) | 0.05% | 0.05% |
| Na₂ EDTA | 0.05% | 0.05% |
| Boric acid | 0.6% | 0.6% |
| Sodium borate | 0.1% | 0.1% |
| Sodium chloride (NaCl) | 0.4% | 0.4% |
| pH | 7.7 | 7.8 |

The above formulations may be prepared by the method described in Example I. In this example, the efficacy of HD in combination with two different concentrations of PHMB (1.5 ppm and 1.0 ppm) as disinfectants was evaluated using a borate buffer system containing 0.4% sodium chloride. Following the Stand-alone test method described in Example 1 (ISO/14729), each solution was evaluated for its antimicrobial efficacy against S. aureus ATCC 6538, P. aeruginosa ATCC 9027, S. marcescens ATCC 13880, Candida albicans ATCC 10231, and Fusarium solani ATCC 36031 after disinfection for 6 hours.

The antimicrobial efficacy of the test solution of Example 2 is illustrated in Table 2.2.

TABLE 2.2

Log reduction at 6 hours

| | Example | Example |
|---|---|---|
| Microorganism | 2A | 2B |
| Staphylococcus aureus ATCC 6538 | 6.2 | 6.2 |
| Pseudomonas aeruginosa ATCC 9027 | 6.0 | 6.0 |
| Serratia marcescens ATCC 13880 | 6.3 | 6.3 |
| Candida albicans ATCC 10231 | 4.23 | 3.97 |
| Fusarium solani ATCC 36031 | 1.4 | 1.46 |

As can be seen, both formulations containing HD completely killed challenge bacterial strains S. aureus, P. aeruginosa and S. marcescens in the presence of low concentration of sodium chloride (0.4%). The both formulations reduced the level of the difficult-to-kill organisms C. albicans and F. solani by approximate 4 and 1.4 log units respectively, exceeding the ISO criterion of at least 1-log reduction of each challenge fungal strain. In addition, the data also suggested that the formulation in a borate buffer system has greater efficacy against fungal strains over the formulation in a phosphate buffer saline (PBS) system.

### Example 3

TABLE 3.1

w/v

| Ingredients | 3A | 3B |
|---|---|---|
| Hexamidine diisethionate (HD) | 0.01% | 0.01% |
| Polyhexamethylene biguanide (PHMB) | 1.5 ppm | 1.0 ppm |
| Poloxamine (Pluronic F87) | 0.05% | 0.05% |
| Na₂ EDTA | 0.05% | 0.05% |
| Boric acid | 0.6% | 0.6% |
| Sodium borate | 0.1% | 0.1% |
| Sodium chloride (NaCl) | 0.2% | 0.2% |
| pH | 7.7 | 7.8 |

The above formulations may be prepared by the method described in Example I. In this example, the efficacy of HD in combination with two different concentrations of PHMB (1.5 ppm and 1.0 ppm) as disinfectants was evaluated using a borate buffer system with a low concentration (0.2%) of sodium chloride. Following the Stand-alone test method described in Example 1 (ISO/14729), each solution was evaluated for its antimicrobial efficacy against the ISO panel organisms (S. aureus ATCC 6538, P. aeruginosa ATCC 9027, S. marcescens ATCC 13880, C. albicans ATCC 10231, and F. solani ATCC 36031), and an Acanthamoeba strain after disinfection for 6 hours as described above.

The antimicrobial efficacy of the test solution of Example 3 is illustrated in Table 3.2.

TABLE 3.2

| | Log reduction at 6 hours | |
|---|---|---|
| | Example | Example |
| Microorganism | 3A | 3B |
| Staphylococcus aureus ATCC 6538 | 6.2 | 6.2 |
| Pseudomonas aeruginosa ATCC 9027 | 6.1 | 6.1 |
| Serratia marcescens ATCC 13880 | 6.3 | 6.3 |
| Candida albicans ATCC 10231 | 6.0 | 6.0 |
| Fusarium solani ATCC 36031 | 2.54 | 2.5 |
| Acanthamoeba polyphaga ROS (MCC 3315) | 3.7 | 2.7 |
| (trophozoites) | | |

As can be seen, both formulations containing HD completely killed challenge strains S. aureus and P. aeruginosa, S. marcescens and C. albicans in the presence of low concentration of sodium chloride (0.2%). Furthermore, both solutions also reduced the difficult-to-kill organism F. solani by 2.5-log units. Interestingly the data demonstrated that the efficacy of formulations using a borate buffer system with 0.2% NaCl (3A and 3B) was superior to the previous formulations in examples 1 and 2 against fungal strains. Further, both formulations (3A and 3B) also reduced A. polyphaga by 3.7 and 2.7-log units respectively.

### Example 4

TABLE 4.1

w/v

| Ingredients | A | B |
|---|---|---|
| Hexamidine diisethionate (HD) | 0.005% | 0.005% |
| Polyhexamethylene biguanide (PHMB) | 1.5 ppm | 1.0 ppm |
| Poloxamine (Pluronic F87) | 0.05% | 0.05% |
| Na₂ EDTA | 0.05% | 0.05% |
| Boric acid | 0.6% | 0.6% |
| Sodium borate | 0.1% | 0.1% |
| Sodium chloride (NaCl) | 0.2% | 0.2% |
| pH | 7.7 | 7.8 |

The above formulation may be prepared by the method described in Example I. In this example, the efficacy of 0.005% of HD in combination with two different concentrations of PHMB (1.5 ppm and 1.0 ppm) as disinfectants was evaluated using a borate buffer system with 0.2% sodium chloride. Following the Stand-alone test method described in Example 1 (ISO/14729), each solution was evaluated for its antimicrobial efficacy against S. aureus ATCC 6538, P. aeruginosa ATCC 9027, S. marcescens ATCC 13880, C. albicans ATCC 10231, F. solani ATCC 36031, and an Acanthamoeba strain after disinfection for 6 hours as described.

The antimicrobial efficacy of the test solution of Example 4 is illustrated in Table 4.2.

TABLE 4.2

| | Log reduction at 6 hours | |
|---|---|---|
| Microorganism | Example 4A | Example 4B |
| Staphylococcus aureus ATCC 6538 | 6.18 | 6.18 |
| Pseudomonas aeruginosa ATCC 9027 | 6.1 | 6.1 |
| Serratia marcescens ATCC 13880 | 4.4 | 5.1 |
| Candida albicans ATCC 10231 | 5.67 | 5.15 |
| Fusarium solani ATCC 36031 | 1.85 | 1.78 |
| Acanthamoeba polyphaga ROS (MCC | 1.7 | 1.7 |
| 3315) (trophozoites) | | |

The formulation containing 0.005% of HD completely killed challenge strains S. aureus and P. aeruginosa, and reduced the level of the difficult-to-kill organism S. marcescens by 4.4 log units. The formulations described in examples 4A and 4B also reduced C. albicans and F. solani by approximate 5.0 and 1.7 log units respectively. Furthermore, both solutions reduced the number of another difficult-to-kill organism A. polyphaga by 1.7 log units.

### Example 5

TABLE 5.1

| | w/v | | |
|---|---|---|---|
| Ingredients | A | B | C |
| Hexamidine diisethionate (HD) | 0.01% | 0.01% | 0.01% |
| Polyhexamethylene biguanide | 1.0 ppm | 1.0 ppm | 1.0 ppm |
| (PHMB) | | | |
| Poloxamine (Pluronic F87) | 0.05% | 0.05% | 0.05% |
| Na₂ EDTA | 0.05% | 0.05% | 0.05% |
| Boric acid | 0.6% | 0.6% | 0.6% |
| Sodium borate | 0.1% | 0.1% | 0.1% |
| Sodium chloride (NaCl) | 0.20% | 0.2% | 0.2% |
| Potassium chloride (KCl) | 0.15% | — | — |
| Di-sodium hydrogen | — | 0.11% | 0.2% |
| orthophosphate (Na₂HPO₄) | | | |
| pH | 7.8 | 7.8 | 7.8 |

The above formulations may be prepared by the method described in Example I. In this example, the efficacy of HD in combination with 1.0 ppm of PHMB as disinfectants was evaluated using a borate buffer system containing 0.2% sodium chloride with different concentrations of di-sodium hydrogen orthophosphate or potassium chloride. Following the Stand-alone test method described in Example 1 (ISO/14729), each solution was evaluated for its antimicrobial efficacy against S. aureus ATCC 6538, P. aeruginosa ATCC 9027, S. marcescens ATCC 13880, C. albicans ATCC 10231, and F. solani ATCC 36031 after disinfection for 6 hours as described above.

The antimicrobial efficacy of the test solution of Example 5 is illustrated in Table 5.2.

TABLE 5.2

| | Log reduction at 6 hours | | |
|---|---|---|---|
| | Example | Example | Example |
| Microorganism | 5A | 5B | 5C |
| Staphylococcus aureus ATCC 6538 | 6.09 | 6.09 | 6.09 |
| Pseudomonas aeruginosa ATCC | 5.84 | 5.84 | 5.84 |
| 9027 | | | |
| Serratia marcescens ATCC 13880 | 6.25 | 5.55 | 6.25 |
| Candida albicans ATCC 10231 | 4.91 | 4.94 | 4.04 |
| Fusarium solani ATCC 36031 | 2.18 | 2.11 | 1.76 |

As can be seen, the 3 formulations containing HD completely killed challenge bacterial strains S. aureus, P. aeruginosa, and S. marcescens in the presence of different concentrations of di-sodium hydrogen orthophosphate or potassium chloride. The formulations described in examples 5A, 5B and 5C reduced the level of the difficult-to-kill organisms C. albicans and F. solani by not less than 4.0 and 1.7 log units respectively, exceeding the ISO criterion of at least 1-log reduction of each challenge fungal strain.

### Example 6

TABLE 6.1

| | w/v | | |
|---|---|---|---|
| Ingredients | A | B | C |
| Hexamidine diisethionate (HD) | 0.005% | 0.005% | 0.005% |
| Polyhexamethylene biguanide | 1.0 ppm | 1.0 ppm | 1.0 ppm |
| (PHMB) | | | |
| Poloxamine (Pluronic F87) | 0.05% | 0.05% | 0.05% |
| Na₂ EDTA | 0.05% | 0.05% | 0.05% |
| Boric acid | 0.6% | 0.6% | 0.6% |
| Sodium borate | 0.1% | 0.1% | 0.1% |
| Sodium chloride (NaCl) | 0.2% | 0.2% | 0.2% |
| Potassium chloride (KCl) | 0.15% | — | — |
| Di-sodium hydrogen | — | 0.11% | 0.2% |
| orthophosphate (Na₂HPO₄) | | | |
| pH | 7.8 | 7.8 | 7.8 |

The above formulations may be prepared by the method described in Example I. In this example, the efficacy of 0.005% HD in combination with 1.0 ppm of PHMB as disinfectants was evaluated using a borate buffer system containing 0.2% sodium chloride with different concentrations of di-sodium hydrogen orthophosphate or potassium chloride. Following the Stand-alone test method described in Example 1 (ISO/14729), each solution was evaluated for its antimicrobial efficacy against S. aureus ATCC 6538, P. aeruginosa ATCC 9027, S. marcescens ATCC 13880, C. albicans ATCC 10231, and F. solani ATCC 36031 after disinfection for 6 hours as described above.

The antimicrobial efficacy of the test solution of Example 6 is illustrated in Table 6.2

TABLE 6.2

| | Log reduction at 6 hours | | |
|---|---|---|---|
| | Example | Example | Example |
| Microorganism | 6A | 6B | 6C |
| Staphylococcus aureus ATCC | 6.09 | 6.09 | 6.09 |
| 6538 | | | |
| Pseudomonas aeruginosa ATCC | 5.84 | 5.84 | 5.84 |
| 9027 | | | |
| Serratia marcescens ATCC 13880 | 5.61 | 4.35 | 6.25 |
| Candida albicans ATCC 10231 | 4.33 | 3.04 | 3.59 |
| Fusarium solani ATCC 36031 | 1.35 | 1.35 | 1.14 |

The formulations containing 0.005% HD completely killed challenge strains S. aureus and P. aeruginosa, and reduced the level of the difficult-to-kill organism S. marcescens by not less than 4.3 log units. The formulations described in examples 6A, 6B and 6C also reduced C. albicans and F. solani by not less than 3.0 and 1.1 log units respectively, exceeding the ISO criterion of at least 1-log reduction of each challenge fungal strain.

### Example 7

This example compares the disinfection efficacy of formulations (Examples 7A and 7B) in this invention with two commercial solutions.

Solution A: A commercial multipurpose disinfection solution containing polyhexanide 0.0001%.

Solution B: A commercial multipurpose disinfection solution containing polyquaterium-1 (PQ-1) 0.001% and myristamidopropyl dimethylamine 0.0005%.

### Example 7A

A combination of hexamidine diisethionate 0.01% and Solution A.

### Example 7B

A combination of hexamidine diisethionate 0.01% and Solution B.

The antimicrobial activity of each solution was evaluated for its antimicrobial efficacy against the ISO panel organisms (S. aureus ATCC 6538, P. aeruginosa ATCC 9027, S. marcescens ATCC 13880, C. albicans ATCC 10231, and F. solani ATCC 36031), an Acanthamoeba strain and two clinical isolates of fungi (C. albicans and F. solani) after disinfection for 6 hours as described above. Comparative disinfection results are given in the Table 7.1.

TABLE 7.1

| | Log reduction at 6 hours | | | |
|---|---|---|---|---|
| | Solution | Example | Solution | Example |
| Microorganism | A | 7A | B | 7B |
| Staphylococcus aureus | 5.20 | 5.99 | 4.55 | 6.01 |
| ATCC 6538 | | | | |
| Pseudomonas aeruginosa | 3.23 | 5.87 | 5.57 | 5.80 |
| ATCC 9027 | | | | |
| Serratia marcescens | 3.17 | 6.03 | 5.15 | 6.15 |
| ATCC 13880 | | | | |
| Candida albicans | 2.16 | 4.24 | 2.02 | 3.40 |
| ATCC 10231 | | | | |
| Fusarium solani | 0.86 | 1.85 | 1.82 | 2.99 |
| ATCC 36031 | | | | |
| Candida albicans | 2.36 | 3.13 | ND | ND |
| GDH 2346* | | | | |
| Candida albicans GRI 682* | 1.01 | 3.56 | ND | ND |
| Fusarium solani 004* | 1.83 | 2.15 | ND | ND |
| Fusarium solani 005* | 1.20 | 2.51 | ND | ND |
| Acanthamoeba polyphaga | 1.00 | 3.71 | ND | ND |
| ROS (MCC 3315) | | | | |
| (trophozoites) | | | | |

ND = not determined;
*clinical isolates

The disinfectant of the present invention in Example 7A and 7B was superior to the polyhexanide containing Solution A and PQ-1 containing Solution B against all the strains tested, particularly with strong activity against difficult-to-kill species of fungi and acanthamoeba.

Additionally, the ability of the disinfectant of the present invention to resist depletion (being taken up into or onto the polymer matrix) by contact lenses was compared with the commercial solution A. A contact lens (Pure Vision) was soaked in 3 mL of each solution for 1 day or 7 days. The solution was subsequently removed into a test tube then challenged with F. solani ATCC 36031. Following the Stand-alone test procedure, the anti-fungal activity of each depleted solution was evaluated. The test results are tabulated in Table 7.2.

TABLE 7.2

| | Log reduction of | |
|---|---|---|
| | F. solani ATCC | |
| | 36031 at 6 hours | |
| | Solution A | Example 7A |
| Stand alone test | 0.86 | 3.44 |
| Stand alone test - Depletion for 1 day | 0.54 | 2.06 |
| Stand alone test - Depletion for 7 days | 0.29 | 1.42 |

The MPDS herein disclosed (Examples 1-7) exhibit antimicrobial efficacy against the panel organisms recommended in the ISO standard (ISO 14729), meeting or exceeding the Stand-alone test criteria of giving more than 3-log reductions against P. aeruginosa (ATCC 9027), S. marcescens (ATCC 13880), S. aureus (ATCC 6538). Furthermore, the compositions also provide high activity against difficult-to-kill species of fungi and Acanthamoeba strains. In addition, when used in combination with polymeric antimicrobials (i.e. PHMB) in contact lens care compositions, the compositions maintain the antimicrobial efficacy after 7 days of depletion with contact lenses.

### Example 8

In this example, three solution formulations listed in Table 8.1 were prepared in distilled water and sterilized by filtering through a 0.22 micron filter. Final concentration of 0.01% HD and 1 ppm PHMB were used as basic active antimicrobial ingredients.

TABLE 8.1

| | Formulations used in the example | | |
|---|---|---|---|
| Component | Example 8A | Example 8B | Example 8C |
| Sodium Chloride (NaCl) | 0.3 | 0.3 | 0.25 |
| EDTA | 0.05 | 0.05 | 0.05 |
| Pluronic | 0.05 | 0.05 | 0.05 |
| Boric acid | 0.6 | 0.6 | 0.6 |
| Sodium borate | 0.1 | 0.1 | 0.1 |
| Potassium dihydrogen | 0.02 | 0 | 0.02 |
| orthophosphate (KH₂PO₄) | | | |
| Di-sodium phosphate | 0.11 | 0 | 0 |
| (anhydrous Na₂HPO₄) | | | |
| PHMB | 1 PPM | 1 PPM | 1 PPM |
| Hexamidine diisethionate | 0.01 | 0.01 | 0.01 |
| (HD) | | | |
| KCl | 0 | 0.1 | 0.1 |
| pH | 7.7 | 7.8 | 7.75 |
| Osmolarity | 229 | 237 | 225 |

Concentrations in % (unless specified)

The antimicrobial efficacy of above three aqueous formulations against the five panel microorganisms (Table 8.2) was evaluated in the presence or absence of organic soil using the standalone method (ISO (14729). An organic soil mixture consisting of heat killed yeast cells (Saccharomyces cerevisiae approximately 1×10⁸ CFU/ml) in inactivated fetal bovine serum (1%) was prepared as described by the ISO 14729.

The antimicrobial activities of the formulations were also evaluated against a range of clinical isolates and Acanthamoeba strains (Table 8.2). Furthermore the effect of the test formulations on Acanthamoeba encystment was examined as described previously (Kilvington 2008).

TABLE 8.2
Microorganisms used in this example

| Microorganism | Source and Phenotype |
|---|---|
| Gram-positive bacteria | |
| Staphylococcus aureus ATCC 6538* | Human isolate |
| Staphylococcus aureus 060 | Hospital strain - MRSA |
| Gram-negative bacteria | |
| Delftia acidovorans 001 | Lens case of asymptomatic |
| | wearer |
| Pseudomonas aeruginosa ATCC 9027* | Otic infection |
| Serratia marcescens ATCC 13880* | Pond water |
| Serratia marcescens 005 | CLARE - MPSR |
| Serratia marcescens 035 | Keratitis - MPSR |
| Stenotrophomonas maltophilia 002 | Lens case of asymptomatic |
| | wearer |
| Yeast | |
| Candida albicans ATCC 10231* | Bronchomycosis |
| Candida albicans GDH 2346 (IER 001) | Denture stomatitis, highly |
| | adherent |
| Candida albicans GRI 682 (IER 002) | Denture stomatitis |
| Fungi | |
| Fusarium solani ATCC 36031* | Corneal ulcer |
| Fusarium solani 004 | Keratitis - MPSR |
| Fusarium solani 005 | Keratitis - MPSR |
| Acanthamoeba | |
| Acanthamoeba castellani ATCC 36868 | Keratitis |
| Acanthamoeba polyphaga ROS (MCC 3315) | Keratitis |

*ISO panel organism;
MRSA, Methicillin-resistant Staphylococcus aureus;
CLARE, Contact lens related acute red eye;
MPSR, Multipurpose solution resistant strain.

As can be seen in Table 8.3, the three example formulations were very active against the panel bacterial strains and met the ISO 14729 criteria giving complete inhibition of growth within 6 h of disinfection time. These three solutions also have high anti fungal activities and achieved >5 and >3 log reductions (in the number of viable microorganisms) against ISO panel C. albicans (ATCC 10231) and F. solani (ATCC 36031) respectively by 6 h of disinfection (Table 8.3).

Similarly, the tested solutions showed a complete killing of three panel bacterial strains in the presence of organic soil after 6 h of disinfection (Table 8.3). The antifungal activity of the tested formulations was attenuated in the presence of organic soil, showing >1.2 and >1.5 log reductions against C. albicans ATCC 10231 and F. solani ATCC 36031 respectively after 6 h of disinfection (Table 8.3), still exceeding the ISO requirement of 1 log reduction.

TABLE 8.3
Average log reduction of viable bacteria after 6 h of
disinfection with three formulations.

| Test | | Example | Example | Example |
|---|---|---|---|---|
| Condition | Bacteria | 8A | 8B | 8C |
| Without | S. aureus ATCC 6538 | 6.1 | 6.1 | 6.1 |
| organic | P. aeruginosa ATCC | 5.9 | 5.9 | 5.9 |
| soil | 9027 | | | |
| | S. marcescens ATCC | 5.9 | 5.3 | 5.8 |
| | 13880 | | | |
| | C. albicans ATCC | 5.52 | 5.87 | 5.87 |
| | 10231 | | | |
| | F. solani ATCC 36031 | 3.12 | 3.19 | 3.19 |
| With | S. aureus ATCC 6538 | 6.00 | 6.00 | ND |
| organic | P. aeruginosa ATCC | 5.60 | 5.60 | ND |
| soil | 9027 | | | |
| | S. marcescens ATCC | 6.00 | 6.10 | ND |
| | 13880 | | | |
| | C. albicans ATCC | 1.26 | 1.32 | ND |
| | 10231 | | | |
| | F. solani ATCC 36031 | 1.59 | 1.64 | ND |

ND, not determined

Moreover, the activities of the three formulations against clinical isolates of bacteria and fungi are presented in Table 8.4. The test formulations exhibited a complete inhibition of growth for two strains of multipurpose solution resistant S. marcescens, and Gram-negative clinical isolates of S. maltophilia and D. acidovorans within 6 h of disinfection time. The three solutions also reduced the difficult-to-kill strains of methicillin-resistant S. aureus by >3 log units after 6 h of disinfection.

Similarly all three formulations also showed excellent disinfectant effect against the difficult-to-kill and highly adherent clinical strains of C. albicans (GDH 2346 and GRI 682) and multipurpose solution resistant strains of F. solani (004 and 005) and displayed >5 and >3 log reductions after 6 h of disinfection respectively (Table 8.4).

TABLE 8.4
Average log reduction of clinical isolates of bacteria and fungi
after 6 h of disinfection with three formulations

| | | Example | Example | Example |
|---|---|---|---|---|
| Microorganism | | 8A | 8B | 8C |
| Bacteria | S. marcescens 035 | 6.06 | 6.06 | 6.06 |
| | S. marcescens 005 | 6.31 | 6.31 | 6.31 |
| | S. maltophilia 002 | 6.15 | 6.15 | 6.15 |
| | D. acidovorans 001 | 5.84 | 5.84 | 5.84 |
| | S. aureus 060 | 4.2 | 4.1 | 5.3 |
| Fungi | C. albicans GDH 2346 | 5.60 | 5.25 | 5.25 |
| | C. albicans GRI 682 | 5.72 | 5.72 | 5.37 |
| | F. solani 004 | 3.20 | 3.29 | 3.18 |
| | F. solani 005 | 3.85 | 3.64 | 3.85 |

The amoebicidal activity of the three formulations is shown in Table 8.5. All three solutions provide high activity against Acanthamoeba strains and achieved 3 log reduction against A. polyphaga ROS (MCC 3315). Formulations 8A and 8B showed 2.6 and 2.8 log reductions respectively against A. castellanii ATCC 30868 after 6 h of disinfection (Table 8.5).

TABLE 8.5
Average log reduction of trophozoites after 6 h of
disinfection with test formulations

| Acanthamoeba strain | Example 8A | Example 8B | Example 8C |
|---|---|---|---|
| A. polyphaga ROS (MCC | 3.0 | 3.0 | 3.0 |
| 3315) | | | |
| A. castellanii ATCC 30868 | 2.6 | 2.8 | ND |

ND, not determined

The trophozoites of A. castellanii ATCC 30868 showed minimal encystment after incubation in the formulations 8A and 8B. The mean value of encystment was 7.6% or 4.2% for Example 8A or Example 8B respectively following 24 h disinfection (Table 8.6) in comparison to approximately 25% of encystment of the test strain in a commercial available multipurpose solution containing PHMB.

TABLE 8.6
Acanthamoeba encystment following trophozoite
incubation (24 h) in example formulations

| | Encystment (%) | | |
|---|---|---|---|
| Acanthamoeba strain | Example 8A | Example 8B | Example 8C |
| A. castellanii ATCC 30868 | 7.6 | 4.2 | ND |

ND, not determined

The current prototypes of new MPDS (Example 8, Table 8.1) have demonstrated (under the stand-alone test procedure recommended in the ISO 14729, after 6 h of disinfection):
Good anti-bacterial activity against three panel bacterial strains (recommended in the ISO 14729) in the presence or absence of organic soil and an ability to exceed the stand-alone test criteria of >3 log reduction, or to give complete inhibition of growth.
Excellent disinfectant activities against the clinically resistant strains of S. marcescens, S. maltophilia, and D. acidovorans with a complete inhibition of growth.
Good activity against the difficult-to-kill strains of methicillin-resistant S. aureus (>3 log units).
Good activity against the ISO (14729) recommended and clinical strains of C. albicans (>5 log reduction) and F. solani (>3 log reduction); exceeding 1-log reduction for fungal strains.
A strong amoebicidal activity against A. castellanii and A. polyphaga, giving >2 log reductions of trophozoites.
Minimal Acanthamoeba encystment following trophozoite incubation (24 h) in example formulations.

The reference to any prior art in this specification is not, and should not be taken as an acknowledgement or any form of suggestion that the referenced prior art forms part of the common general knowledge in Australia.

### REFERENCES CITED HEREIN

Alizadeh, H., Silvany, R. E., Meyer, D. R., Dougherty, J. M. and McCulley, J. P. (1997) In vitro amoebicidal activity of propamidine and pentamidine isethionate against Acanthamoeba species and toxicity to corneal tissues. Cornea 16, 94-100.
Bourcier, T., Thomas, F., Borderie, V., Chaumeil, C. and Laroche, L. (2003) Bacterial keratitis: predisposing factors, clinical and microbiological review of 300 cases. Br J Ophthalmol 87, 834-838.
Brasseur, G., Favennec, L., Perrine, D., Chenu, J. P. and Brasseur, P. (1994) Successful treatment of Acanthamoeba keratitis by hexamidine. Cornea 13, 459-462.
Budavari, S. (1989) The Merck Index. An Encyclopedia of Chemicals, Drugs, and Biologicals. Rahway, N.J., U.S.A: MERCK & CO.
Cano-Parra, J., Bueno-Gimeno, I., Lainez, B., Cordoba, J. and Montes-Mico, R. (1999) Antibacterial and antifungal effects of soft contact lens disinfection solutions. Cont Lens Anterior Eye 22, 83-86.
Cheng, K. H., Leung, S. L., Hoekman, H. W., Beekhuis, W. H., Mulder, P. G., Geerards, A. J. and Kijlstra, A. (1999) Incidence of contact-lens-associated microbial keratitis and its related morbidity. Lancet 354, 181-185.
Dannelly, H. K. and Waworuntu, R. V. (2004) Effectiveness of contact lens disinfectants after lens storage. Eye Contact Lens 30, 163-165.
Dart, J. K., Stapleton, F. and Minassian, D. (1991) Contact lenses and other risk factors in microbial keratitis. Lancet 338, 650-653.
Dutot, M., Paillet, H., Chaumeil, C., Warnet, J. M. and Rat, P. (2009) Severe ocular infections with contact lens: role of multipurpose solutions. Eye 23, 470-476.
Epand, R. M. and Epand, R. F. (2009a) Domains in bacterial membranes and the action of antimicrobial agents. Mol Biosyst 5, 580-587.
Epand, R. M. and Epand, R. F. (2009b) Lipid domains in bacterial membranes and the action of antimicrobial agents. Biochim Biophys Acta 1788, 289-294.
Gilbert, P. and Moore, L. E. (2005) Cationic antiseptics: diversity of action under a common epithet. Journal of Applied Microbiology 99, 703-715.
Grare, M., Dibama, H. M., Lafosse, S., Ribon, A., Mourer, M., Regnouf-de-Vains, J. B., Finance, C. and Duval, R. E. (2009) Cationic compounds with activity against multidrug-resistant bacteria: interest of a new compound compared with two older antiseptics, hexamidine and chlorhexidine. Clinical Microbiology and Infection.
Hume, E. B., Zhu, H., Cole, N., Huynh, C., Lam, S. and Willcox, M. D. (2007) Efficacy of contact lens multipurpose solutions against serratia marcescens. Optom V is Sci 84, 316-320.
Ide, T., Miller, D., Alfonso, E. C. and O'Brien, T. P. (2008) Impact of contact lens group on antifungal efficacy of multipurpose disinfecting contact lens solutions. Eye Contact Lens 34, 151-159.
John, T., Lin, J. and Sahm, D. F. (1990) Acanthamoeba keratitis successfully treated with prolonged propamidine isethionate and neomycin-polymyxin-gramicidin. Ann Ophthalmol 22, 20-23.
Keay, L., Stapleton, F. and Schein, O. (2007) Epidemiology of contact lens-related inflammation and microbial keratitis: a 20-year perspective. Eye Contact Lens 33, 346-353, discussion 362-343.
Perrine, D., Chenu, J. P., Georges, P., Lancelot, J. C., Saturnino, C. and Robba, M. (1995) Amoebicidal efficiencies of various diamidines against two strains of Acanthamoeba polyphaga. Antimicrobial Agents and Chemotheraphy 39, 339-342.
Review, T. C. I. (2007) Cosmetic Ingredient safety Review cosmetic Expert Panel
Richards, M. R. and Xing, D. K. (1994) Investigation of synergism with combinations of dibromopropamidine isethionate or propamidine isethionate and polymyxin B. Journal of Pharmacy and Pharmacology 46, 563-566.
Seal, D. V. (2003) Acanthamoeba keratitis update-incidence, molecular epidemiology and new drugs for treatment. Eye 17, 893-905.
Standardization, I.O.S. (2001) Ophthalmic optics—Contact lens care products. Microbiological requirements and test methods for products and regimens for hygienic management of contact lenses. In ISO 14729 ed. Standardization, I.O.S.
Toxicol, I. J. (2007) Final Report on the Safety Assessment of Hexamidine and Hexamidine Diisethionate International Journal of Toxicology 26, 79-88.
Vasseneix, C., Gargala, G., Francois, A., Hellot, M. F., Duclos, C., Muraine, M., Benichou, J., Ballet, J. J., Brasseur, G. and Favennec, L. (2006) A keratitis rat model for evaluation of anti-Acanthamoeba polyphaga agents. Cornea 25, 597-602.
Wien, R., Harrison, J. and Freeman, W. A. (1948) Diamidines as antibacterial compounds. British Journal of Pharmacology and Chemotheraphy 3, 211-218.
Wright, P., Warhurst, D. and Jones, B. R. (1985) Acanthamoeba keratitis successfully treated medically. Br J Ophthalmol 69, 778-782.

1. A multi-purpose disinfecting solution for disinfecting and cleaning contact lenses, said solution comprising a compound or any acceptable salt, functional variant, derivative or analog thereof wherein the compound has the formula (II): 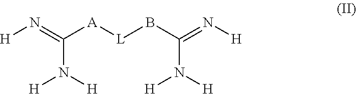
wherein A and B are each independently selected from the group consisting of C, N, O, optionally substituted C₃-C₁₂ cycloalkyl, optionally substituted C₆-C₁₈ aryl, optionally substituted C₁-C₁₈ heteroalkyl and optionally substituted C₁-C₁₈ heteroaryl; and
L is selected from the group consisting of H, OH, NO₂, CN, NH₂, halogen, optionally substituted C₁-C₁₂ alkyl, optionally substituted C₂-C₁₂ alkenyl, optionally substituted C₂-C₁₂ alkynyl, optionally substituted C₃-C₁₂ cycloalkyl, optionally substituted C₃-C₁₂ cycloalkenyl, optionally substituted C₁-C₁₀ heteroalkyl, optionally substituted C₂-C₁₂ heterocycloalkyl, optionally substituted C₂-C₁₂ heterocycloalkenyl, optionally substituted C₆-C₁₈ aryl, optionally substituted C₁-C₁₈ heteroaryl, optionally substituted C₁-C₁₂ alkyloxy, optionally substituted C₁-C₁₂ alkyldioxy optionally substituted C₂-C₁₂ alkenyloxy, optionally substituted C₂-C₁₂ alkenyldioxy, optionally substituted C₂-C₁₂ alkynyloxy, optionally substituted C₂-C₁₂ alkynyldioxy, optionally substituted C₃-C₁₂ cycloalkyloxy, optionally substituted C₃-C₁₂ cycloalkenyloxy, optionally substituted C₁-C₁₀ heteroalkyloxy, optionally substituted C₁-C₁₂ heterocycloalkyloxy, optionally substituted C₁-C₁₂ heterocycloalkenyloxy, optionally substituted C₆-C₁₈ aryloxy, optionally substituted C₁-C₁₈ heteroaryloxy, and optionally substituted C₁-C₁₂ alkylamino.

2. The solution of claim 1 wherein A and B are each optionally substituted C₆ aryl.

3. The solution of claim 1 wherein L is optionally substituted C₁-C₁₂ alkyl.

4. The solution of claim 1 wherein the derivative of the compound is any isomer and/or tautomer.

5. The solution of claim 4 wherein said isomer and/or tautomer is selected from the group comprising organic acids, mineral acids and their salts.

6. The solution of claim 5 wherein the organic acid is sulfonic acid or carboxylic acid.

7. The solution of claim 1 wherein the compound has the formula (III): 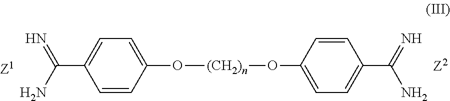
wherein n is 1, 2, 3, 4, 5, 6, 7, 8, 9, 10, 11 or 12, Z¹ and Z² are the same or different and are selected from an organic acid, its salt or a combination thereof.

8. The solution of claim 7 wherein Z¹ and Z² are selected from the group consisting of Isethionate, Methanesulfonate or both.

9. The solution of claim 8 wherein Z¹ and Z² are the same.

10-14. (canceled)

15. The solution of claim 1 wherein the maximum concentration of said compound in said solution is not toxic to ATCC L929 murine fibroblast according to ISO 10993-5 standard procedure for medical device cytotoxicity assessment.

16-22. (canceled)

23. The solution of claim 15 comprising an effective amount of the compound to inhibit the growth of or to kill one or more microorganisms.

24. The solution of claim 23 further comprising a second compound.

25. The solution of claim 24 wherein the second compound is selected from the group consisting of biguanides, myristamidopropyl dimethylamine, and polyquaternary ammonium compounds.

26. The solution of claim 25 wherein the biguanides are selected from the group consisting of salts of alexidine, alexidine free base, salt of chlorhexidine, hexamethylene biguanides and polymeric biguanides.

27. The solution of claim 25 wherein the polyquaternary ammonium compounds are POLYQUAD.

28-36. (canceled)

37. A lens case comprising a compound or any acceptable salt, functional variant, derivative or analog thereof which has the formula (II) of claim 1.

38-41. (canceled)

42. A kit comprising:
the multi-purpose disinfecting solution as claimed in claim 1; and
instructions for using the multi-purpose disinfecting solution in a lens or storage case for multiple-use contact lenses.

43. A method of disinfecting or preserving a multiple-use contact lens comprising contacting the lens with the multi-purpose disinfecting solution as claimed in claim 1.

44-50. (canceled)

51. The solution of claim 3 wherein the optionally substituted C₁-C₁₂ alkyl is a C₃-C₉ alkyl.

52. The solution of claim 26 wherein the polymeric biguanides are polyhexamethylene biguanide (PHMB).
